(19) United States
(12) Patent Application Publication
(10) Pub. No.: US 2015/0088155 A1
(43) Pub. Date: Mar. 26, 2015

(54) MECHANICAL CONFIGURATIONS FOR A MULTI-SITE LEADLESS PACEMAKER

(71) Applicant: Cardiac Pacemakers, Inc., St. Paul, MN (US)

(72) Inventors: Jeffrey E. Stahmann, Ramsey, MN (US); Arthur J. Foster, Blaine, MN (US); William J. Linder, Golden Valley, MN (US); Keith R. Maile, New Brighton, MN (US)

(21) Appl. No.: 14/479,587

(22) Filed: Sep. 8, 2014

Related U.S. Application Data
(60) Provisional application No. 61/881,026, filed on Sep. 23, 2013.

(51) Int. Cl. A61N 1/375; A61N 1/05

(52) U.S. Cl. CPC A61N 1/3756; A61N 1/056; A61N 1/0587
USPC 606/129

(57) ABSTRACT

Devices or methods such as for stimulating excitable tissue or sensing physiologic response or other signals are described. An implantable apparatus can comprise an electrostimulation electrode assembly that can include an electrostimulation unit, at least first and second electrodes, and a fixation guide. The electrostimulation unit can generate electrostimulation for stimulating excitable tissue to achieve desired diagnostic or therapeutic effects. The first and second electrodes, coupled to the electrostimulation unit, can deliver the electrostimulation to two or more stimulation sites such as inside two or more heart chambers or on the surface of the heart. The fixation guide can engage and retain a maneuvering device used for steerably positioning and securing the first and second electrostimulation electrodes at respective stimulation site.

### CLAIM OF PRIORITY

This application claims the benefit of priority under 35 U.S.C. §119(e) of U.S. Provisional Patent Application Ser. No. 61/881,026, filed on Sep. 23, 2013, which is herein incorporated by reference in its entirety.

### TECHNICAL FIELD

This document relates generally to medical devices, and more particularly, to devices and methods for stimulating excitable tissue or sensing physiologic response.

### BACKGROUND

Ambulatory medical devices, such as implantable pacemakers and cardioverter-defibrillators, can chronically stimulate excitable tissues or organs, such as a heart, such as to treat abnormal cardiac rhythms such as bradycardia or tachycardia, or to help improve cardiac performance such as by correcting cardiac dyssynchrony in a patient with congestive heart failure (CHF). Such ambulatory medical devices can have at least first and second electrodes that can be positioned within the heart or on a surface of the heart for contacting the cardiac tissue. The electrodes can be electrically coupled to an electronics unit such as a pulse generator, such as via a lead, and can be used to deliver one or more electrostimulations to the heart, such as to improve or to restore the normal heart function.

### OVERVIEW

Cardiac stimulation using an implantable medical device (IMD) can involve one or more implantable leads that can be transvascularly inserted into one of the heart chambers, such as an atrium or a ventricle. Stimulation of the heart can be accomplished through direct myocardium stimulation using at least first and second electrodes that can be electrically connected to the IMD and in close contact with the cardiac tissue. The electrodes can be positioned along the one or more implantable leads. The stimulation can be provided at specified stimulation strength (e.g., stimulation energy) sufficient to capture the heart tissue, resulting in an evoked electrical depolarization and mechanical contraction.

The stimulation electrodes can be placed inside a heart. The lead can have an insulated electrical conductor or conductors for connecting the IMD to the electrodes positioned in the heart. Connection between the IMD and the lead can be achieved by using a set of matched connectors, which can be respectively located at a header of the IMD and a proximal end of a lead. A threaded setscrew, such as can be located on the header of the IMD, can be used to mechanically affix the proximal end of the lead to the IMD, or to establish an electrical connection between the IMD and the electrodes.

An IMD system using one or more leads can present certain issues during its use in a patient. For example, the connection between the leads and the IMD can be increasingly complicated when more leads need to be connected to the IMD configured for multi-chamber pacing. The lead-IMD connection can malfunction such as via disconnection, erosion of a setscrew, insulation damage, or conductor breakage, among others. Such connection malfunction can lead to sensing failure, inappropriate delivery of electrostimulations, or inappropriate withholding of electrostimulation therapy. Additionally, the leads can be a major source of complication associated with the IMD implantation. Moreover, the IMD, when located subcutaneously in a patient, can present a bulge in the skin that can be less cosmetically appealing. The IMD can even cause irritation, extrusion, or infection—particularly when patients subconsciously or obsessively manipulate or twiddle the IMD.

Self-contained or leadless IMDs, such as a pacemaker, a defibrillator, or a neurostimulator, have been proposed to overcome some of the issues associated with the lead-based IMDs. With the absence of long leads and the complicated system connection between the leads and the IMD, the self-contained IMD may pose fewer complications and can be more cosmetic for patient. For example, a self-contained or leadless pacemaker can be implanted inside a heart. However, due to its size and the lack of leads, these self-contained leadless systems may be only able to pace from only one site or one chamber of the heart, and may lack the flexibility for selective multi-site or multi-chamber pacing as compared to the lead-based IMD system. Additionally, some patients, including children or persons with a compromised venous system, may benefit clinically from electrostimulation using at least first and second electrodes affixed on the heart surface such as for epicardial stimulation. In sum, the present inventors have recognized that there still remains a considerable need for medical devices and methods of using such devices such as for improving flexibility and reliability of chronic cardiac stimulation therapy.

Various embodiments described herein can help improve multi-site electrical cardiac stimulation. For example, an implantable apparatus can comprise an electrostimulation electrode assembly. The electrostimulation electrode assembly can be implanted inside a heart or on a heart surface. The electrostimulation electrode assembly can include an electrostimulation unit, two or more electrodes, and at least one fixation guide. The electrostimulation unit can include a housing and an electrostimulation generation circuit configured to generate electrostimulations for stimulating an excitable tissue. The two or more electrodes, such at least first and second electrodes, can be coupled to the electrostimulation unit and configured to be capable of independently delivering respective electrostimulations to respective separate first and second stimulation sites on or in the excitable tissue, such as two or more sites inside two or more heart chambers or on the surface of the heart. The at least one fixation guide can be included in or coupled to the housing of the electrostimulation. The fixation guide can receive and retain a respective maneuvering device, and permit using the maneuvering device to position the electrostimulation electrode assembly at a target implant location. The fixation guide can allow the maneuvering device to steerably position and secure the first electrode to the first stimulation site, and to steerably position and secure the second electrode to the second stimulation site when the first electrode has been secured to the first stimulation site.

A method example can include providing an implantable medical device (IMD) configured to generate electrostimulation for stimulating two or more stimulation sites, such as first and second stimulation sites inside or on a surface of a heart of a subject via two or more electrodes. The IMD can be positioned at a target implant location, which includes providing a temporary, at least partially elongated delivery member configured to attach to the IMD. The delivery member can then be attached to the IMD, and the attached delivery member and the IMD can be disposed to the target implant location. The method includes providing a fixation element on the IMD. The fixation element can be configured to affix the at least first and second electrodes to the first and second stimulation sites using at least one fixation guide that can receive and retain the maneuvering device for positioning the IMD at the target implant location. The method includes releasing the delivery member from the IMD, and delivering the electrostimulation to the two or more stimulation sites via the two or more electrodes.

This Overview is an overview of some of the teachings of the present application and not intended to be an exclusive or exhaustive treatment of the present subject matter. Further details about the present subject matter are found in the detailed description and appended claims. Other aspects of the invention will be apparent to persons skilled in the art upon reading and understanding the following detailed description and viewing the drawings that form a part thereof, each of which are not to be taken in a limiting sense. The scope of the present invention is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates a schematic example of an implantable apparatus that includes an electrostimulation electrode assembly.

FIGS. 2A-D illustrate examples of configurations of an implantable electrostimulation electrode assembly with at least one extended electrode.

FIGS. 3A-B illustrate an example of an electrostimulation unit with one or more fixation guide.

FIG. 4 illustrates an example of a delivery member used for delivering an electrostimulation unit and positioning the extended electrostimulation electrodes.

FIGS. 5A-C illustrate examples of electrostimulation electrode assembly implanted inside one or more heart chambers.

FIGS. 6A-B illustrate examples of electrostimulation electrode assembly implanted on the heart surface.

FIGS. 7A-C illustrate examples of electrostimulation electrodes assembly with electrodes attached to an exterior of the electrostimulation unit.

FIGS. 8A-B illustrate examples of electrostimulation electrode assembly implanted inside or outside the heart.

FIG. 9 illustrates an example of a method for stimulating a target issue in a body.

FIG. 10 illustrates an example of a method for disposing and affixing an implantable medical device to the target location of the heart.

### DETAILED DESCRIPTION

Disclosed herein are apparatuses and methods for stimulating a target tissue of a heart or other excitable tissues of a patient, such as to achieve desired diagnostic or therapeutic effect. The target tissue can include heart tissue inside one or more heart chambers (e.g., endocardium) or heart tissue on an outer surface of the heart (e.g., epicardium). The stimulation apparatus can include one or more electrostimulation electrodes that can be affixed to the target tissue using a fixation member. The apparatus and the methods described herein can also be applicable to stimulation or sensing of other tissues or organs in the body.

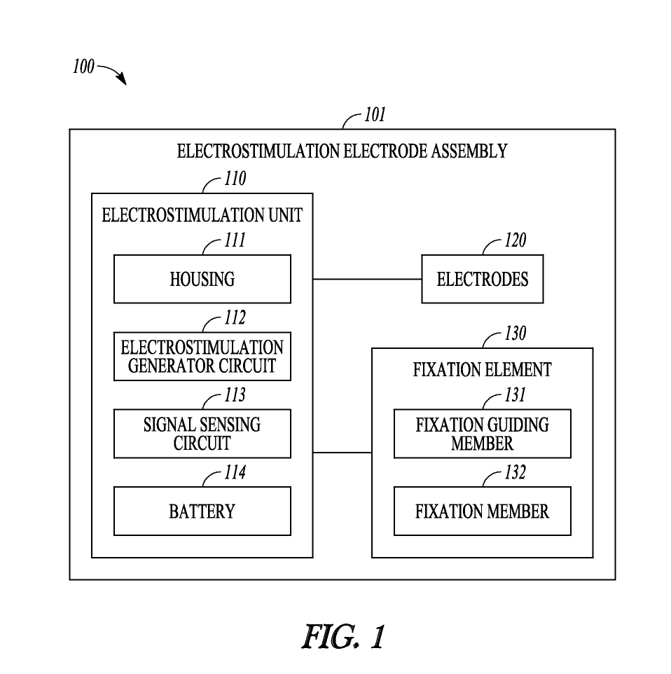

FIG. 1 illustrates a schematic example of an implantable apparatus 100 that can include an electrostimulation electrode assembly 101. The electrostimulation electrode assembly 101 can be configured to be chronically affixed to a location of a heart such as on the surface of the heart or inside a heart chamber such as an atrium or a ventricle, and to provide chronic stimulation to the heart to achieve desirable diagnostic or therapeutic effects. Alternatively or additionally, the electrostimulation electrode assembly 101 can be affixed to other tissues or organs and deliver electrostimulations therein. Examples of such tissues or organs can include an interior or exterior of an artery or vein, a nerve bundle, skin, a carotid body, a stomach or intestine, a bladder, a kidney, soft tissue, gastric tissue, or neural tissue.

The electrostimulation electrode assembly 101 can include an electrostimulation unit 110, at least first and second electrodes 120, and a fixation element 130. The at least first and second electrodes 120 and the fixation element 130 can be coupled to the electrostimulation unit 110.

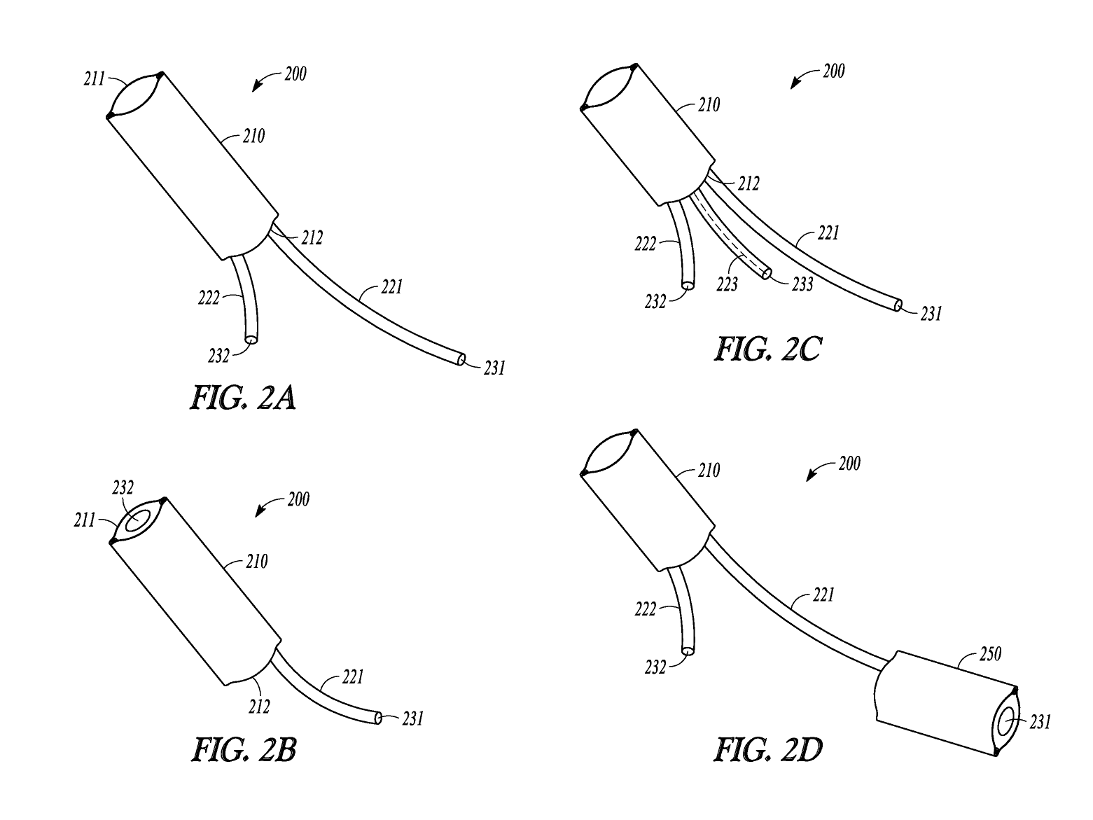

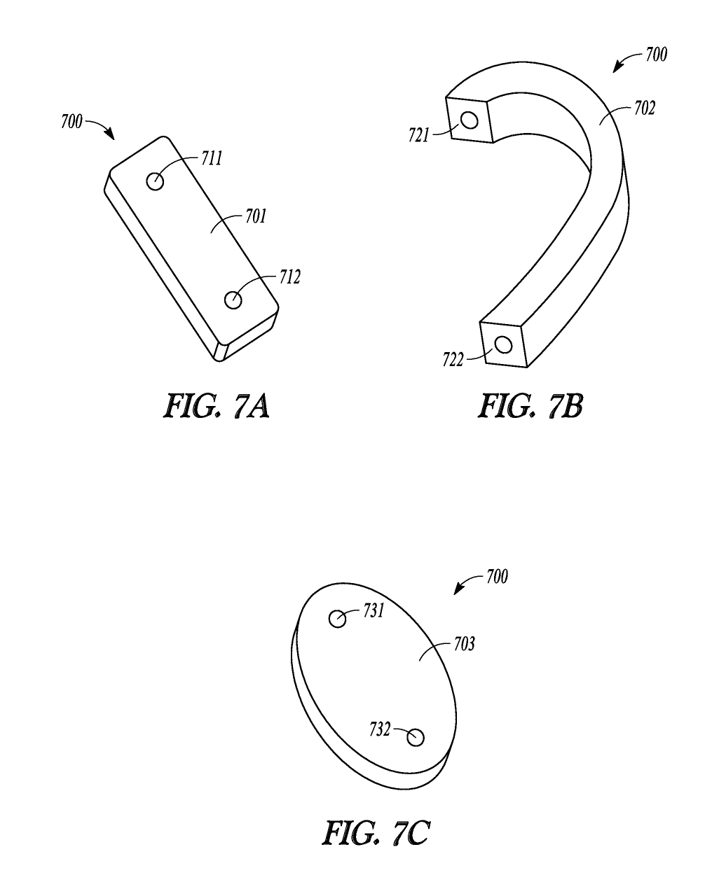

The electrostimulation unit 110 can include a housing 111, an electrostimulation generator circuit 112, and a battery 114. Optionally, the electrostimulation unit 110 can further include a signal sensing circuit 113. The housing 111 can enclose the electrostimulation generator circuit 112, the battery 114, and the signal sensing circuit 113. The housing 111 can be sized and shaped to be chronically implanted at or near the stimulation site such as on the surface of the heart, inside a heart chamber, within a blood vessel, within subcutaneous tissue, within myocardium, or at other locations in the body of a patient. The housing 111 can be sized and shaped such as to allow the at least first and second electrodes 120, which can be coupled to the electrostimulation unit 110, to be in close contact with two or more sites of an excitable tissue, such as endocardial or epicardial sites of two or more heart chambers. The housing 111 can be shaped to facilitate the delivery, positioning, or retaining of the electrostimulation unit 110 at a target implant location, such as inside the heart or on the heart surface. For example, the housing 111 have a shape of a cylinder, a round or oval disk, U-shaped, or others, such as illustrated in FIGS. 2A-2D and 7A-7C.

The electrostimulation generator circuit 112, enclosed within the housing 111, can include circuitry configured to generate electrostimulation pulses. The electrostimulation generator circuit 112 can include circuitry for communicating with a programming device, receiving command such as programming parameters from the programming device, and generating electrostimulation pulses according to the received programming parameters. The electrostimulation pulses, when delivered to the target tissue, can restore or improve a physiologic function, or provide other therapeutic functions, including anti-bradycardia pacing, anti-tachycardia pacing, cardioversion, defibrillation, cardiac resynchronization therapy, neural modulation, among others. The electrostimulation pulses can also be used for diagnosing a disease or a disease condition such as by sensing and analyzing local tissue responses or systemic responses to the delivered electrostimulation pulses.

The signal sensing circuit 113 can be configured to sense at least one physiologic signal from the patient. The sensed physiologic signal can include biopotential sensed from the tissue at or near the at least first and second electrodes 120. The signal sensing circuit 113 can be configured to process the sensed biopotential or physiological signals and generate signal metrics indicative of diagnostics or therapy efficacy. The generated signal metrics can be presented to an end-user such as via a user-interface communicating with the signal sensing circuit 113. The sensed physiologic signals can be provided to the electrostimulation generation circuit 112 to adaptively adjust the programming parameters such as to deliver electrostimulations with desired strength and duration to the target tissue.

The battery 114 can provide power to the operation of the electrostimulation generation circuit 112 and the signal sensing circuit 113. In an example, the battery 114 can include a rechargeable battery configured to be rechargeable such as by using a charging device. The charging device can be external to the patient and can wirelessly communicate with the battery 114 such as using an inductive, an acoustic, or other communication links. In another example, the battery 114 can be recharged using an energy harvesting device within the electrostimulation unit 110. The energy harvesting device can include biomechanical, piezoelectric, or photovoltaic devices configured to harvest thermal, kinetic, or other types of energy generated by body motion, breathing, body temperature, blood pressure, or other physiologic activities or processes.

The at least first and second electrodes 120, coupled to the electrostimulation unit 110, can be configured to contact at least first and second stimulation sites including epicardial or endocardial sites of two or more heart chambers, and to deliver the electrostimulation thereto or therein. The electrode 120 can be an on-housing electrode such as affixed on an exterior of the housing 111. The electrode 120 can be extended electrodes that can be connected to the housing 111 via an extension cable. With the extension cable, the extended electrode can be adapted to stimulate excitable tissues at a distance away from the housing 111. Examples of the extension electrodes and the on-housing electrodes are illustrated in FIGS. 2A-2D and FIGS. 7A-7C, respectively.

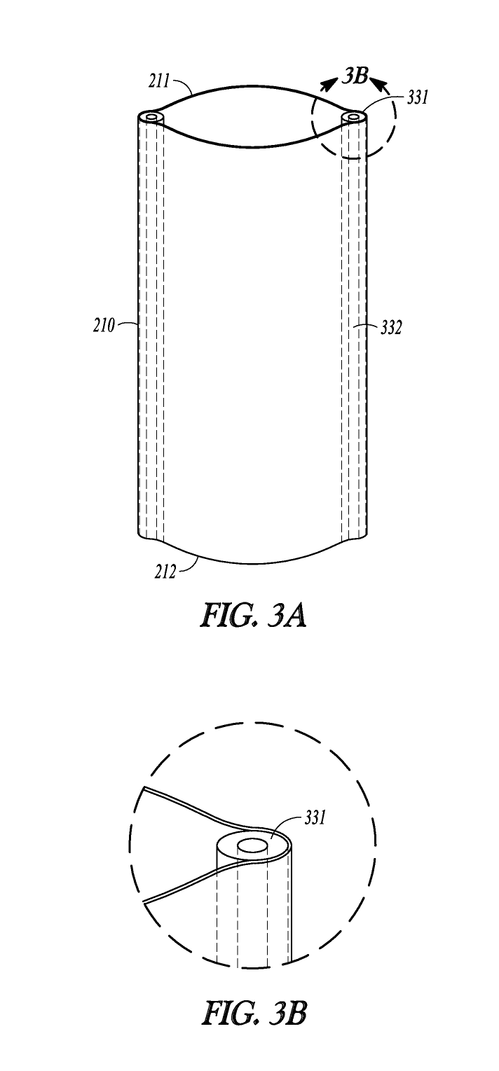

The fixation element 130 can include at least one fixation guide 131 and at least one fixation member 132. The at least one fixation guide 131 can be sized, shaped or otherwise configured to receive and retain a respective maneuvering device, such as a guide wire or a stylet, for steerably positioning the electrostimulation electrode assembly 101 at a target implant location, such as a location at or near the two or more stimulation sites inside the heart or on the heart surface. The fixation guide 131 can be affixed to the electrostimulation unit 110 such as on an exterior or an interior of the housing 111, such that when the maneuvering device is engaged with the fixation guide 131, the electrostimulation unit 110 can be steered by an end-user and positioned to the target implant location. The fixation guide 131 can also allow the maneuvering device to steerably position and secure the first electrode to the first stimulation site, and to steerably position and secure the second electrode to the second stimulation site when the first electrode has been secured to the first stimulation site. Examples of the fixation guide 131 are discussed below, such as with reference to FIGS. 3A-B and 4.

The fixation member 132 can be configured to affix the electrostimulation electrode assembly to a desired implant location inside a heart or on a heart surface. The fixation member 132 can be mounted on various mounting positions on or along the electrostimulation electrode assembly 101. For example, the fixation member 132 can be mounted on an exterior surface of the housing 111. In an example when the electrode 120 is an extended electrode connected to the electrostimulation unit 110 via an extension cable, such as those illustrated in FIGS. 2A-D, the fixation member 132 can be mounted on the distal end of the extension cable in close vicinity to the electrostimulation electrode.

The fixation member 132 can include a passive fixation mechanism, an active fixation mechanism, or a combination of one or more passive or active fixation mechanisms. Examples of the passive fixation can include one or more tines, one or more fins, one or more helices, or one or more other extension structures. Examples of the active fixation can include one or more screws, one or more hooks, one or more barbs, one or more helices, or one or more other tissue-penetrating mechanisms. In an example, the fixation member 132 can include a rotationally-oriented element, such as a helical coil or a spiral coil. The fixation member 132 can include a tapered end on the rotationally-oriented element to facilitate active engagement with the target tissue, such as a screw-in helical coil. The fixation member 132 can be made out of biocompatible materials with desired stiffness, such as stainless steel, titanium alloy, polymer, ceramic, or other synthetic biocompatible metallic or non-metallic materials. In some examples, the fixation member 132 can be electrically coupled to the electrostimulation generator circuit 112 or the signal sensing circuit 113, such that the fixation member 132 can deliver electrostimulations to the target tissue or to sense a biopotential or a physiological signal from the tissue at the fixation site.

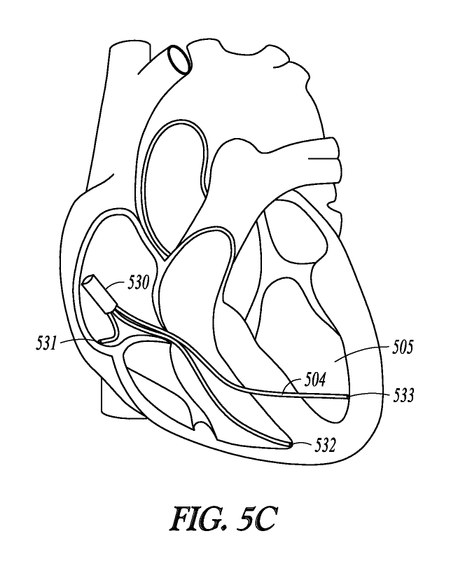

FIGS. 2A-D illustrate various examples of configurations of an implantable electrostimulation electrode assemblies 200 with at least one extended electrode. The implantable electrostimulation electrode assembly 200 can be an embodiment of the electrostimulation electrode assembly 101. The implantable electrostimulation electrode assemblies 200 can comprise an electrostimulation unit 210 and two or more electrodes such as 231, 232, or 233. At least one of the electrodes can be an extended electrode that is coupled to the electrostimulation unit 210 via an elongated extension cable such as 221, 222, or 223. Although only one electrode is disposed on an extension cable as illustrated in FIGS. 2A-D, two or more electrodes can be attached to a common extension cable. The housing 111 can also be configured to include one or more electrodes. The various configurations as illustrated in FIGS. 2A-D can be used in accordance with the anatomy of the stimulation site and the target location for implanting the electrostimulation electrode assembly 200. Examples of steerably positioning the extended electrodes for intracardiac or epicardial electrostimulation are discussed below, such as with reference to FIGS. 5A-C and 6A-B.

FIG. 2A illustrates an example of an implantable electrostimulation electrode assembly 200 that includes two extended electrodes 231 and 232 each electrically connected to the electrostimulation unit 210 through a respective extension cable 221 or 222. The electrostimulation unit 210 can include a cylindrical shaped housing that carries electrostimulation generation circuit, battery, and other electronics and components. The electrostimulation unit 210 can have a proximal end 211 and a distal end 212. The extension cables 221 and 222 can be permanently attached to the electrostimulation unit 210, such as extend from the distal end 212 of the electrostimulation unit 210. In an example, the extension cables 221 and 222 can each include a shielded wire such as encapsulated within the extension cable. The shielded wire can establish communication between the extended electrodes 231 and 232 and the electrostimulation unit 210, deliver electrostimulation pulses to the connected electrodes, or transmit the sensed biopotential or physiologic signals from the electrode to the signal sensing circuit 113.

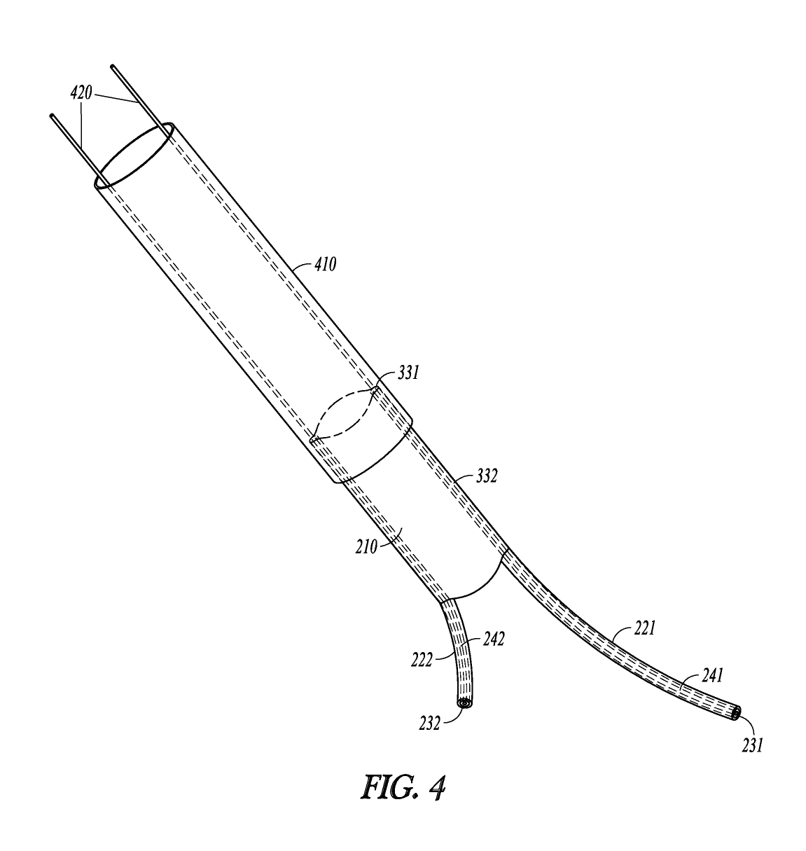

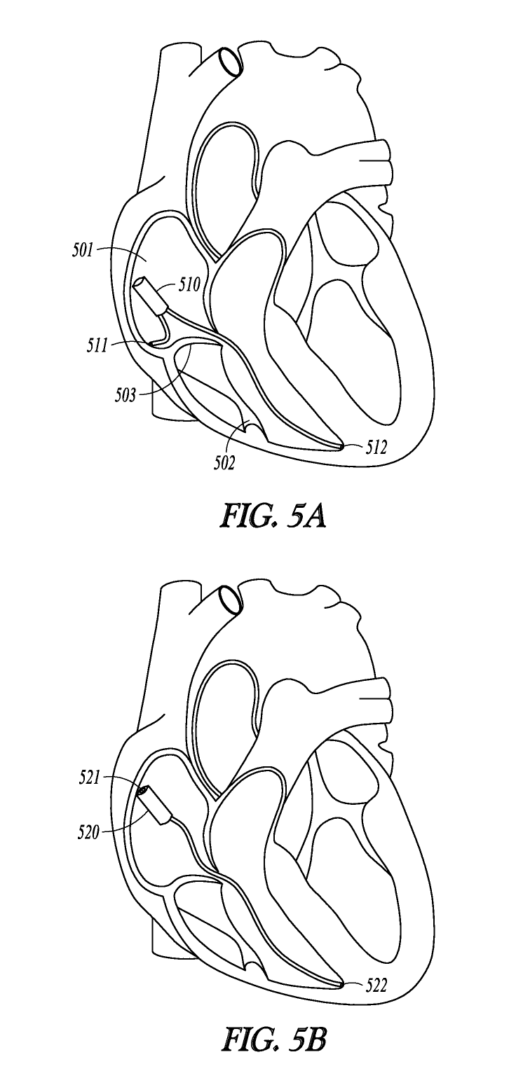

The extension cables 221 and 222 can be in a form of a lead, a catheter, or other types of longitudinal apparatus made of at least electrically conducive materials. At least a portion of the extension cables 221 and 222, such as an exterior portion of each cable, can be made out of biocompatible materials with desired elasticity, durability, or other mechanical properties. The extension cable can include a lumen configured for permitting a maneuvering device, such as a stylet or a guide wire, to pass through and to engage the respective electrodes 231 and 232 disposed at the distal end of the extension cable, and to facilitate the steerable positioning of the electrodes to desired stimulation sites such as inside the heart or on the heart surface. The electrodes 231 and 232 can be sized, shaped, or otherwise configured to be affixed to the target tissue using either a passive or active fixation mechanism. Alternatively, a separate fixation member, such as the fixation member 132 as previously discussed with reference to FIG. 1, can be mounted in close vicinity of the respective electrode. Examples of the extended electrodes and the positioning of the electrode through a maneuvering device through the lumen of the extension cable are discussed below, such as with reference to FIG. 4.

The extension cables 221 and 222 can have different dimensions, such as length, to allow for easy access to two or more spatially separated stimulation sites. As illustrated in FIG. 2A, the electrostimulation unit 210 can be positioned at an implant location inside the heart or on the heart surface. The extended electrode 232 attached to the shorter extension cable 222 can be positioned on an endocardial site inside the right atrium (RA), while the extended electrode 231 attached to the longer extension cable 221 can be positioned on an endocardial site inside the right ventricle (RV). As such, the implantable electrostimulation electrode assembly 200 can be used to achieve dual-chamber stimulation using the extended electrodes 231 and 232.

FIG. 2B illustrates an example of a configuration of the implantable electrostimulation electrode assembly 200 that includes a first extended electrode 231 and a second on-housing electrode 232. The first extended electrode 231 can be coupled to the electrostimulation unit 210 via an extension cable 221 permanently connected to the distal end 212 of the electrostimulation unit 210. The second on-housing electrode 232 can be mounted directly on or along an exterior of the housing of the electrostimulation unit 210 without using an extension cable, such as on the proximal end 211 of the electrostimulation unit 210 as illustrated in FIG. 2B. The on-housing electrode 232 can be shaped and configured to securely anchor the electrostimulation unit 210 to the target implant location.

FIG. 2C illustrates an example of the implantable electrostimulation electrode assembly 200 that includes three extended electrodes 231, 232 and 233, each connected to the electrostimulation unit 210 via respective extension cables 221, 222, and 223. The electrostimulation electrode assembly 200 can be used to stimulate three different sites inside the heart or on the heart surface, such as by placing the extended electrodes 221 through 223 on three different heart chambers including RA, RV and left ventricle (LV). Alternatively, one or more of the sites can be in or on a single heart chamber. The extended electrodes 221 through 223 can be extended from the distal end 212 of the electrostimulation unit 210. In some examples, the extended electrodes 231 through 233 can be distributively coupled to the electrostimulation unit 210 on different locations, including distal end, proximal end, or anywhere along the exterior of the electrostimulation unit 210.

FIG. 2D illustrates an example of a configuration of the implantable electrostimulation electrode assembly 200 that includes a first electrostimulation unit 210 and a second electrostimulation unit 250. The electrostimulation units 210 and 250 can be interconnected such as by an extension cable 221. The first electrostimulation unit 210 can be a primary electrostimulation unit and the second electrostimulation unit 250 can be an auxiliary electrostimulation unit. The implantable electrostimulation electrode assembly 200 can include two electrostimulation electrodes 231 and 232, where the electrode 232 can be an extended electrode coupled to the first electrostimulation unit 210 via an extension cable 222; and the electrode 231 can be an on-housing electrode mounted on an exterior of the second electrostimulation unit 210, such as on the distal end as illustrated in FIG. 2D. The second electrostimulation unit 250 can be configured to generate electrostimulation for stimulating at least one stimulation site different from the site from which the first electrostimulation unit 210 is configured to stimulate. In an example, the first electrostimulation unit 210 can be configured to stimulate a first stimulation site on or in a first heart chamber via the extended electrode 232, and the second electrostimulation unit 250 can be configured to stimulate a second stimulation site on or in a second heart chamber via the on-housing electrode 231.

The electrostimulation units 210 and 250 can be electrically coupled to and communicated with each other such as via a wire or other electrically conductive material within the extension cable 221. At least one of the electrostimulation units 210 and 250 can comprise a power supply configured to provide power to both the electrostimulation units 210 and 250. With the charged batteries of the two or more electrostimulation units, the power sharing mechanism between the interconnected electrostimulation units 210 and 250 can extend the life of the implantable electrostimulation electrode assembly 200. It can also provide more flexible stimulation to multiple sites such as in the heart chambers or on the heart surface.

FIG. 3A illustrates an example of an electrostimulation unit 210 with one or more fixation guide for use with an implantable electrostimulation electrode assembly, such as the assembly 200 illustrated in FIGS. 2A-D. The fixation guide can be included in or coupled to an at least partially elongated housing of the electrostimulation unit. The fixation guide can include one or more bores 331 and respective one or more hollow channels 332. FIG. 3B illustrates a magnified perspective of a local region around the one or more bores 331.

The one or more bores 331 can be disposed on the exterior or the interior of the cylindrical shaped housing of electrostimulation unit 210. The bores 331 and the hollow channels 332 can be bulged from the exterior of the housing of the electrostimulation unit 210. The openings of the bores 331 can have round, oval, polygon, or other shapes than can permit easy access by a maneuvering device such as a stylet.

The openings of the bores can be flat and situated at the same level as the surface of the proximal end 211 (as shown in FIG. 3A) or distal end 212 of the electrostimulation unit 210. In some examples, the opening of the bores can protrude above, or recess below the surface of the proximal end 211 of the electrostimulation unit 210. As an alternative to the flat bore surface, or in combination therewith, the one or more bores 331 can have a cone or funnel shaped opening on at least one of the proximal end 211 or a distal end 212 of the electrostimulation unit 210. The opening can have a specified funnel angle to facilitate the reception and insertion of the maneuvering device into the bores and the retaining of the maneuvering device within the connecting hollow channel 332. The inserted maneuvering device, such as a guide wire, a stylet, or a catheter, can be used to steer and position the electrostimulation unit 210 to a target implant location. The inserted maneuvering device can also engage an extended electrode, such as those illustrated in FIGS. 2A-D, and steerably position the extended electrode to a desirable stimulation site. Examples of the using a maneuvering device to maneuver the electrostimulation unit 210 and to engage an extended electrode are discussed below such as with reference to FIG. 4.

When two or more bores 331 are included in the electrostimulation unit 210, such as illustrated in FIG. 3A, each bore can include a unique identifier to distinguish it from the other bores. Examples of the bore identifiers can include different bore size, shape, color painted on or near the bores, or any combination thereof. The bore identifier can allow a user to insert a selected maneuvering device to an identified bore to steerably position the electrostimulation unit 210 at a target implant location, or to steerably position the extended electrodes to desirable stimulation sites.

The one or more bores 331 can connect to respective one or more hollow channels 332 configured to receive and retain the maneuvering device inserted through the bores 331. The hollow channels 332 can be in parallel to an axle of the cylindrical shaped housing of the electrostimulation unit 210. The one or more bores and the respective one or more hollow channels can each include on its interior surface a coating such as of one or more tissue ingrowth-inhibition materials, such as expanded Polytetrafluoroethylene (ePTFE). Other examples of the tissue ingrowth-inhibition material can include electro-spun polyurethane or micro-textured material.

FIG. 4 illustrates an example of a delivery member 410 that can be used for delivering an electrostimulation unit 210 and positioning the extended electrostimulation electrodes 231 and 232. The delivery member 410 can be a temporary tool configured to facilitate delivery of the electrostimulation electrode unit 210 to the target implant location. The delivery member 410 can be used transvascularly or through a surgically prepared subcutaneous tunnel during implantation of the electrostimulation unit 210. The delivery member 410 can also be used to reposition one or more electrodes 231 and 232 such as to different stimulation sites, or to explant or extract the electrostimulation unit 210 from the implant location.

The delivery member 410 can be sized, shaped, or otherwise configured to allow secure attachment to the electrostimulation electrode unit 210 during implantation or easy detachment from the electrostimulation electrode unit 210 during explantation. As illustrated in FIG. 4, the delivery member 410 can have an at least partially elongated, such as cylindrical shaped body coaxial with the cylindrical shaped electrostimulation electrode unit 210. The diameter of the cross-section of the delivery member 410 can be larger than the diameter of the cylindrical shaped electrostimulation electrode unit 210, such that at least a portion of the electrostimulation electrode unit 210 can be inserted within a portion of the delivery member 410. The delivery member 410 and the electrostimulation electrode unit 210 can each include a respective detachable coupler feature to secure the attachment between the electrostimulation electrode unit 210 and the delivery member 410, and to detach the delivery member 410 easily from the electrostimulation electrode unit 210. The coupler features can be mounted on the exterior surface of electrostimulation electrode unit 210 and the interior surface of the delivery member 410. Examples of the coupler features can include a snap-fit coupling, a threaded or other rotation or screw-in coupling, a slide-in engagement, or one or more other locking mechanisms.

The two extended electrodes 231 and 232 can be connected to the electrostimulation electrode unit 210 via respective extension cables 221 and 222. The extension cables can each include a respective lumen 241 and 242 configured for permitting a maneuvering device 420 to pass through and to engage the extended electrodes attached to the distal end of the extension cable. The one or more bores 331 and the hollow channels 332 on the electrostimulation electrode unit 210 can be aligned with the lumen 241 and 242 of the respective extension cables 221 and 222. The alignment as such can permit the maneuvering device 420 to pass through the bore 331, the hollow channel 332, and the lumen 241, and to engage and steer the extended electrode 231. Using the maneuvering device 420, an end-user can position the electrostimulation unit 210 at a target implant location, and to steerably position the electrode 231 and 232 on desired stimulation sites such as inside the heart or on the heart surface. For example, the maneuvering device 420 can be used to steerably position and secure the first extended electrode 231 to the first endocardial stimulation site in the RV, and to steerably position and secure the second extended electrode 232 to the second endocardial stimulation site in the RA.

Each electrostimulation electrode can correspond to a unique bore such as on the housing of the electrostimulation unit 210. The identifiers keyed to different bores can allow an end-user to easily identify the electrode to be positioned. For example, the bore corresponding to the extended electrode 231 can have a unique identifier such as a unique size, shape, color code, or any combination thereof, which is different than the identifier of the bore corresponding to electrode 232. The end-user can rely on the bore identifiers to selectively engage, steerably position, and secure the extended electrode 231 to an endocardial RV stimulation site, and to selectively engage, steerably position, and secure the extended electrode 232 to an endocardial RA stimulation site.

FIGS. 5A-C illustrate examples of various configurations of electrostimulation electrode assembly totally implanted inside one or more heart chambers. The various configurations of the electrostimulation electrode assembly can be examples of the electrostimulation electrode assembly 101, or any of the electrostimulation electrode assembly 200 as discussed previously with reference to FIGS. 2A-D. The electrostimulation electrode assemblies as shown FIGS. 5A-C can be implanted inside the heart such as using the delivery member 410 through a transvascular approach. The implanted electrostimulation electrode assembly can provide programmed electrostimulation to multiple sites of two or more heart chambers. The electrostimulation can correct pathological heart rhythms, restore or improve heart functions, or to achieve other therapeutic purposes. The implanted electrostimulation electrode assembly can also sense the biopotentials or physiologic signals from inside the heart such as to provide diagnostic information to the end-user.

FIG. 5A illustrates an example of an electrostimulation electrode assembly with two extended electrodes 511 and 512. The electrostimulation unit 510 can be positioned in the right atrium (RA) 501. A first shorter extended electrode 511 can be positioned on a site inside the RA 501, while a second longer extended electrode 512 can be advanced across the tricuspid valve 503 and be positioned on a site inside the right ventricle (RV) 502, such as on the apex of the RV. The electrodes 511 and 512 can be sized and shaped to include a fixation member to actively or passively affix to the intracardiac tissue. A dedicated fixation member can be included on or along the extension cable, such as at the distal end of the extension cable. The implanted electrostimulation electrode assembly can provide programmed endocardial electrostimulation to the RA and the RV through the respective extended electrodes.

FIG. 5B illustrates an example of an electrostimulation electrode assembly with two electrodes 521 and 522. The electrostimulation unit 520 can be positioned in the RA 501. A first electrode 521 can be an on-housing electrode mounted on the exterior surface of the electrostimulation unit 520, and can deliver electrostimulations to an endocardial site inside the RA 501. A second electrode 512 can be an extended electrode with a extension cable long enough to be advanced across the tricuspid valve 503 and be positioned on an endocardial site inside the RV 502, such as on the apex of the RV. Because the on-housing electrode 521 is attached to the housing of the electrostimulation unit 520, by affixing the electrode 521 to the RA tissue, the electrostimulation unit 520 can be securely anchored to the target implant location in the RA.

FIG. 5C illustrates an example of an electrostimulation electrode assembly with three extended electrodes 531, 532, and 533. The electrostimulation unit 530 can be positioned in the RA 501. A first extended electrode 531 can be positioned on an endocardial site inside the RA 501. A second extended electrode 532 can be advanced across the tricuspid valve 503 and be positioned on an endocardial site inside the RV 502, such as on the apex of the RV. A third extended electrode 533 can be advanced through a tunnel 504 surgically prepared in the septum, and be positioned in an endocardial site inside the LV 505. As such, the implanted electrostimulation electrode assembly can provide multi-chamber programmed electrostimulation to the RA, RV and LV through the respective extended electrodes. The multi-chamber electrostimulation can achieve desired therapy regimes such as cardiac resynchronization therapy (CRT) to treat patients with congestive heart failure.

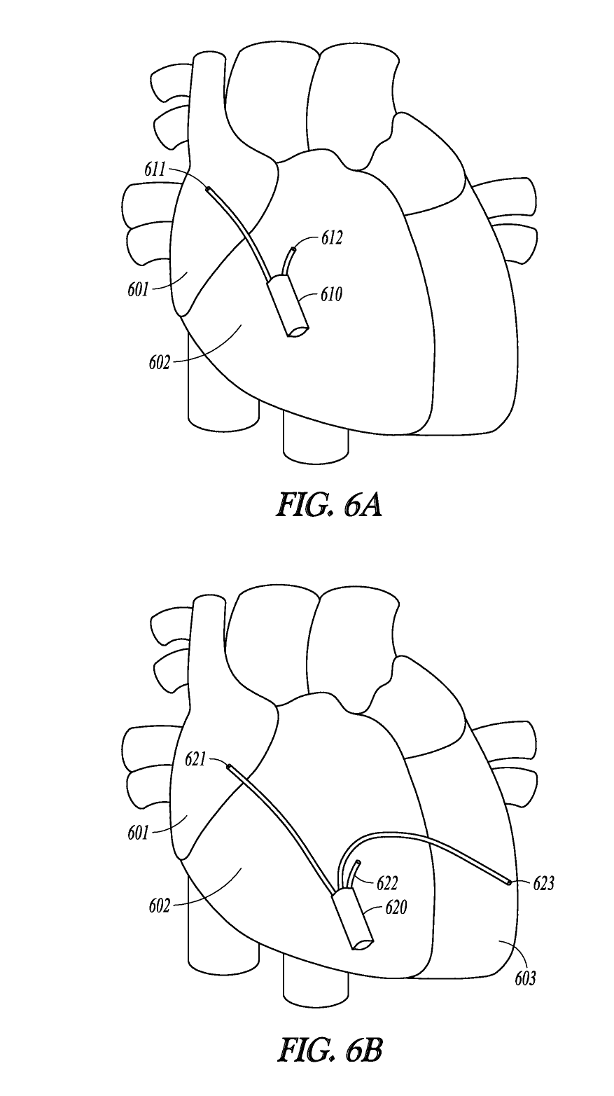

FIGS. 6A-B illustrate examples of various configurations of electrostimulation electrode assembly totally implanted outside the heart such as on the heart surface. The various configurations of the electrostimulation electrode assembly can be examples of the electrostimulation electrode assembly 101, or any of the electrostimulation electrode assembly 200 as discussed previously with reference to FIGS. 2A-D. The electrostimulation electrode assembly as shown FIGS. 6A-B can be implanted such as using the delivery member 410 through a subcutaneous approach. The implanted electrostimulation electrode assembly can provide programmed electrostimulation to multiple sites of one or more heart chambers. The electrostimulation can correct pathological heart rhythms, restore or improve heart functions, or to achieve other therapeutic purposes. The implanted electrostimulation electrode assembly can also sense the biopotentials or physiologic signals from outside the heart using at least the extended electrodes such as to provide diagnostic information to the end-user.

FIG. 6A illustrates an example of an electrostimulation electrode assembly with two extended electrodes 611 and 612. The electrostimulation unit 610 can be positioned at or near the heart surface, such as a location between an epicardial surface of the RA and an epicardial surface of the RV. A first extended electrode 611 can be positioned on an epicardial site on the RA 601, while a second extended electrode 612 can be positioned on an epicardial site on the RV 602. The electrodes 611 and 612 can be sized or shaped to include a fixation member that can actively or passively affix the respective electrode on the epicardial tissue. A dedicated fixation member can be included on or along the extension cable, such as at the distal end of the extension cable, to affix the electrode to the target epicardial tissue. The implanted electrostimulation electrode assembly can provide programmed electrostimulation to the RA and RV through the respective extended electrodes.

FIG. 6B illustrates an example of an electrostimulation electrode assembly with three extended electrodes 621, 622, and 623. The electrostimulation unit 620 can be positioned at or near the heart surface, such as a location near the epicardial surface of the RV. A first extended electrode 621 can be positioned on an epicardial site on the RA 601. A second extended electrode 622 can be positioned on an epicardial site on the RV 602. A third extended electrode 623 can be positioned on an epicardial site on the LV 603. The implanted electrostimulation electrode assembly can provide multi-chamber programmed electrostimulation to the RA, RV and the LV through the respective extended electrodes. The multi-chamber programmed electrostimulation can achieve desired therapy regimes such as CRT to treat patients with congestive heart failure.

FIGS. 7A-C illustrate examples of various configurations of electrostimulation electrodes assemblies 700 with on-housing electrodes attached to an exterior of the electrostimulation unit housing. The electrostimulation electrodes assemblies 700 can include an electrostimulation unit (such as 701, 702, or 703) and one or more on-housing electrostimulation electrodes (such as 711 and 712, 721 and 722, or 731 and 732) coupled to the electrostimulation unit. The electrostimulation unit can be sized, shaped, or otherwise configured for permitting the at least one on-housing electrode to contact a desired stimulation site such as inside a heart chamber or on a heart surface. All or part of the electrostimulation unit can be flexible to facilitate implantation, fixation or other property of the unit.

The electrostimulation unit can include a disk-shaped or a U-shaped three dimensional structure with at least one flat surface. The flat surface can have a round, oval, or square shape on which at least one on-housing electrode can be mounted. As illustrated in FIG. 7A, a pair of on-housing electrodes 711 and 712 can be mounted on a rectangular shaped flat surface on an exterior of a disk-shaped electrostimulation unit 701. FIG. 7B illustrates a U-shaped electrostimulation unit 702. The two on-housing electrodes 721 and 722 can each be affixed to a flat surface on the distal ends of the two arms of the electrostimulation unit 702. In FIG. 7C, a pair of on-housing electrodes 731 and 732 can be mounted on a round or oval shaped flat surface of a disk-shaped electrostimulation unit 703. Electrostimulation unit of other three dimensional structure with surfaces of various shapes for mounting one or more electrostimulation electrodes have also been contemplated, and they are within the scope of the present discussion of the electrostimulation electrodes assemblies 700.

The electrostimulation electrodes assemblies 700 can include one or more fixation guides on or along the exterior of the electrostimulation electrodes unit. Examples of the fixation guides can include one or more bores 331 the corresponding linked hollow channels 332, such as those illustrated in FIGS. 3A-B. The bores can be configured to allow a maneuvering device, such as a stylet or a guiding wire, to engage and steerably position the electrostimulation electrodes unit to a target implant location, and to secure the electrodes on the electrostimulation electrodes unit to the desired stimulation sites.

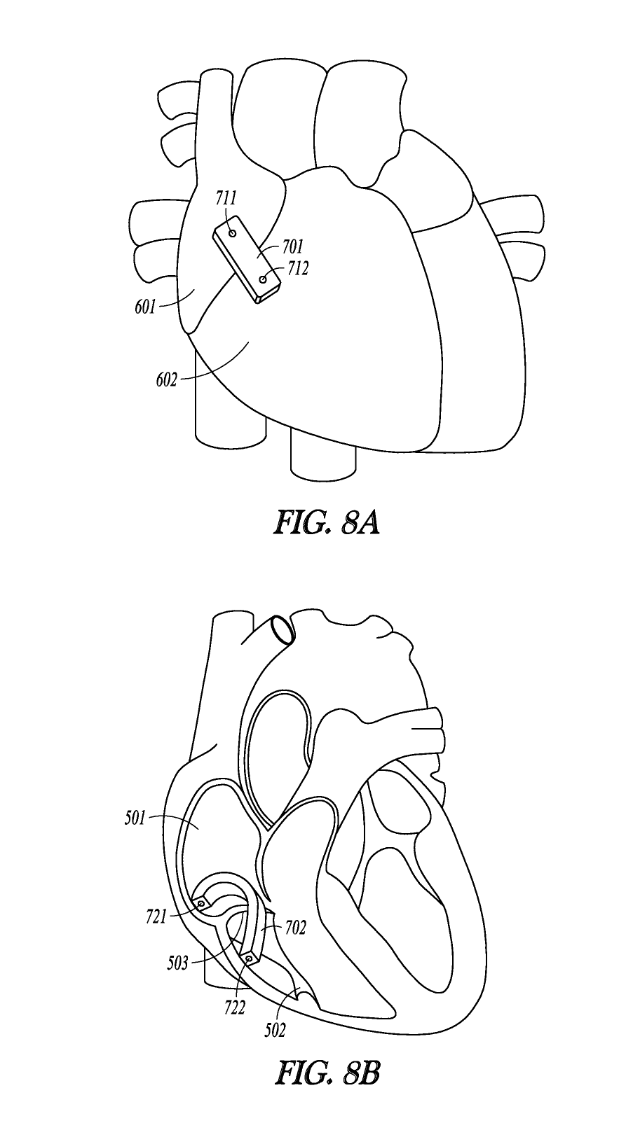

FIGS. 8A-B illustrate examples of various configurations of electrostimulation electrode assembly totally implanted inside or outside the heart. The various configurations of the electrostimulation electrode assembly can be examples of the electrostimulation electrode assembly 101, or any of the electrostimulation electrode assembly as discussed previously with reference to FIGS. 7A-C. The electrostimulation electrode assembly as shown in FIGS. 8A-B can be implanted such as using the delivery member 410 through a subcutaneous or transvascular approach. The implanted electrostimulation electrode assembly can provide programmed electrostimulation to multiple sites of two or more heart chambers. The electrostimulation can correct pathological heart rhythms, restore or improve heart functions, or to achieve other therapeutic purposes. The implanted electrostimulation electrode assembly can also sense the biopotentials or physiologic signals from inside the heart using at least the extended electrodes such as to provide diagnostic information to the end-user.

FIG. 8A illustrates a disk-shaped electrostimulation electrode unit 701 implanted on the epicardial surface of a heart. The electrostimulation electrode unit 701 is positioned such that the first on-housing electrode 711 can contact an epicardial site on the RA 601, and the second on-housing electrode 712 can contact an epicardial site of the RV 602. Such placement can allow the electrostimulation electrode unit 701 to deliver programmed stimulation to the RA 601 and the RV 602 to achieve desired therapeutic effects.

FIG. 8B illustrates a U-shaped electrostimulation electrode unit 702, implanted inside the heart. The electrostimulation electrode unit 702 can be steerably advanced across the tricuspid valve 503, such that the first arm of the electrostimulation electrode unit 702 can be disposed inside the RA 501, and that the second arm of the electrostimulation electrode unit 702 can be disposed inside the RV 502. The on-housing electrode 721 on the first arm can contact an intracardiac site inside the RA 501, and the on-housing electrode 722 on the second arm can contact an intracardiac side inside the RV 502. Such placement can allow the U-shaped electrostimulation electrode unit to deliver programmed stimulation to the RA and RV to achieve desired therapeutic effects.

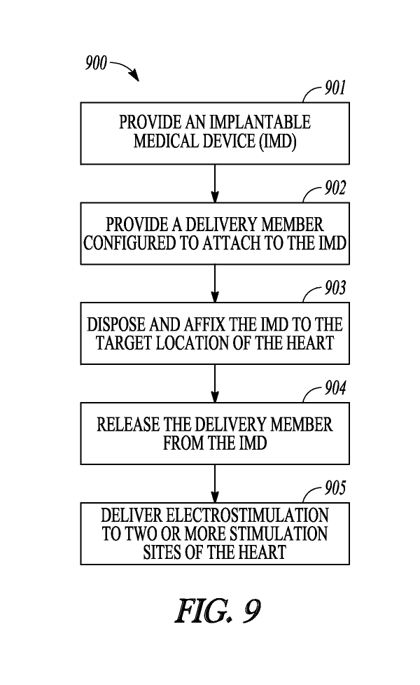

FIG. 9 illustrates an example of a method 900 for stimulating a target issue in a body, such as a site inside a heart chamber or an epicardial surface of the heart. The method 900 can be performed such as using the electrostimulation electrode assembly 100, one or more electrostimulation electrode assembly 200 that includes a plurality of extended electrodes such as those illustrated in FIGS. 2A-D, or one or more electrostimulation electrode assembly that includes a plurality of electrodes on an surface of the housing of the electrostimulation electrode unit such as those illustrated in FIGS. 7A-C.

At 901, an implantable medical device (IMD) is provided for use to stimulate a stimulation site such as to achieve a desired diagnostic or therapeutic effect. The stimulation can be delivered to an exterior (such as on the heart surface) or an interior (such as inside heart chambers) of a heart to treat heart rhythm disorders or to restore or improve cardiac function such as in patients with congestive heart failure (CHF). Examples of the electrostimulation for such purposes can include temporary or chronic bradycardia pacing, tachycardia pacing, cardioversion or defibrillation shock, or cardiac resynchronized pacing for treating CHF. In various examples, the IMD can be configured to deliver electrostimulations to other tissues or organs including an interior or exterior of an artery or vein, a nerve bundle, skin, a carotid body, a stomach or intestine, a bladder, a kidney, soft tissues, gastric tissues, or neural tissues.

In an example, providing the IMD includes providing an electrostimulation electrode assembly which can include a plurality of extended electrodes such as those illustrated in FIGS. 2A-D or a plurality of on-housing electrodes on the surface of the electrostimulation electrode assembly such as those illustrated in FIGS. 7A-C. The plurality of electrodes can be configured to be positioned in two or more different sites in the heart or in other organs to tissues for multi-site stimulation.

At 902, a delivery member is provided for use to attach to the IMD. The delivery member, such as the delivery member 410 as previously discussed with reference to FIG. 4, can have a size and shape such as to encompass at least a portion of the IMD, such as a part of the IMD housing. The delivery member and the IMD can each include a coupler feature to allow the delivery member to detachably engage with the IMD.

At 903, the attached IMD and the delivery member can be disposed to a desired implantation site such as the inside of the heart or the heart surface. The delivery member can be shaped to facilitate the delivery of the attached IMD transvascularly or through a surgically prepared subcutaneous tunnel. When the attached IMD and the delivery member are positioned to the desired implant site, the plurality of electrodes on the IMD can be affixed to the target site of the tissue. A fixture member can be provided at or near the electrostimulation electrode, such as on the exterior of the IMD or on a distal end of an extension cable connected to the IMD. A maneuvering device, such as a stylet or a guide wire, can be inserted through a fixation guide attached to the exterior or inside the IMD. The maneuvering device can engage the electrodes and steerably position the electrode to the excitable tissue at or around the desired stimulation sites. The fixation member can be configured to be actively affixed to a target tissue, such as by using a screw-in helical or spiral coil, or other rotationally-oriented active fixation element. In some examples, the fixation member can be passively affixed to a target tissue.

When the IMD is positioned at the target implant location and the electrodes be steerably positioned and affixed to the desired stimulation site, at 904 the delivery member can be released and detached from the IMD. This can be achieved, for example, by disengaging the coupler features on the IMD and the delivery member. At 905, electrostimulation can be delivered to two or more stimulation sites via the two or more implanted electrodes affixed at the stimulation sites.

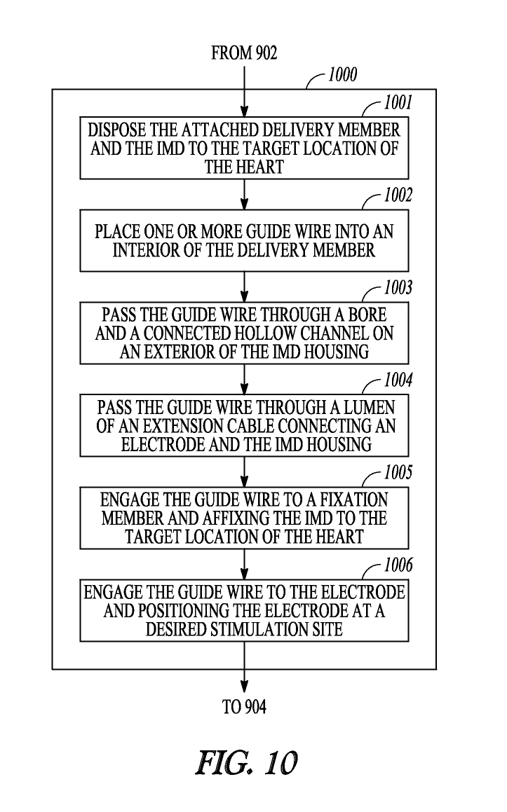

FIG. 10 illustrates an example of a method 1000 for disposing and affixing the IMD to the target location of the heart. The method 1000 can be an example of element 903 in affixing the IMD to the desired stimulation site in the heart.

At 1001, the attached delivery member and the IMD can be disposed to a target intracardiac implant location transvascularly, or be disposed to a target epicardial implant location through a surgically prepared subcutaneous tunnel. At 1002, one or more maneuvering devices, such as a stylet or a guide wire, can be placed inside the delivery member. Then, at 1003, the maneuvering device can be inserted and passed through a fixation guide attached to the IMD. The fixation guide, such as illustrated in FIGS. 3A-B and 4, can receive and retain the maneuvering device. The fixation guide can include one or more bores linking to respective one or more hollow channels parallel to an axle of an at least partially elongated housing of the IMD. Through the fixation guide, the maneuvering device can be used to engage the IMD and steerably position the IMD to a target implant location inside the heart or on the heart surface.

At 1004, the maneuvering device can be advanced to pass through a lumen of an extension cable. The extension cable can connect an electrode and the IMD housing. Examples of such extended electrodes with extension cables can include those discussed with reference to FIGS. 2A-D.

At 1005, the maneuvering device can engage with a fixation member, such as a screw-in helix, or an electrode sized and shaped to facilitate fixation such as a screw-in electrode. An end-user can operate the engaged maneuvering device and steerably position the IMD to the target implant location. At 1006, the maneuvering device can be used to steerably position the electrostimulation electrode such that the electrode can closely contact the excitable tissue at the stimulation site. Testing of the electrical stimulation can then be performed, such as by measuring evoked tissue response following delivery of electrostimulation pulses. The evoked response can include, for example, tissue stimulation threshold, sensed electrogram signal strength, or tissue impedance. When the desired fixation and electrode positioning are achieved, such as the evoked response being within a specified range, the electrodes can be permanently secured to the stimulation sites, and the delivery member can be released from the IMD and retrieved, as shown at 904.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A human-implantable apparatus comprising:
an epicardially or endocardially implantable electrostimulation electrode assembly, including:
an electrostimulation unit, including a housing carrying an electrostimulation generation circuit, the electrostimulation generation circuit configured to generate electrostimulations for stimulating an excitable tissue;
at least first and second electrodes, coupled to the electrostimulation unit, the at least first and second electrodes configured to be capable of independently delivering respective electrostimulations to respective separate first and second stimulation sites on or in the excitable tissue; and
at least one fixation guide, included in or coupled to the housing of the electrostimulation unit, the at least one fixation guide configured:
to receive a maneuvering device;
to permit using the maneuvering device to position the electrostimulation electrode assembly at a target implant location; and
to permit using the maneuvering device to steerably position and secure the first electrode to the first stimulation site, and to steerably position and secure the second electrode to the second stimulation site when the first electrode has been secured to the first stimulation site.

2. The apparatus of claim 1, comprising at least one extension cable permanently attached to the electrostimulation unit, wherein at least one of the first and second electrodes is configured to be attached to the extension cable and electrically connected to the electrostimulation unit through the extension cable.

3. The apparatus of claim 2, wherein the extension cable includes a lumen configured for permitting the maneuvering device to pass through and to position the at least one of the first and second electrodes to at least one of the first and the second stimulation sites.

4. The apparatus of claim 1, wherein:
at least one of the first and the second electrodes is configured to attach to an exterior of the housing of the electrostimulation unit; and
the electrostimulation unit is sized, shaped, or otherwise configured to permit the at least one of the first and the second electrodes to contact one of the first and the second stimulation sites.

5. The apparatus of claim 1, wherein the at least one fixation guide includes one or more bores on the housing of the electrostimulation unit.

6. The apparatus of claim 5, wherein the at least one fixation guide includes one or more hollow channels along an at least partially elongated housing of the electrostimulation unit, the one or more hollow channels configured to connect to the one or more bores.

7. The apparatus of claim 5, wherein the one or more bores or the one or more hollow channels each has an interior surface treated with one or more tissue ingrowth-inhibition materials.

8. The apparatus of claim 5, wherein the one or more bores each has a funnel shaped member sized or configured to receive the maneuvering device.

9. The apparatus of claim 5, wherein the one or more bores each includes an identifier configured to distinguish one bore from the other bores.

10. The apparatus of claim 5, wherein at least one of the bores and at least one of the hollow channels are configured to connect to a lumen of the extension cable, and to allow the maneuvering device to pass through the at least one bore, the at least one hollow channel, and the lumen of the extension cable, and to position the at least one electrode attached to the extension cable to one of the first and second stimulation sites.

11. The apparatus of claim 1, wherein the electrostimulation unit includes a signal sensing circuit coupled to at least one of the first and second electrodes, the signal sensing circuit configured to sense and analyze at least one physiologic signal from the patient.

12. The apparatus of claim 1, comprising at least one auxiliary electrostimulation unit electrically coupled to the electrostimulation unit, the auxiliary electrostimulation unit configured to generate electrostimulation for stimulating one or more stimulation sites of the excitable tissue.

13. The apparatus of claim 12, wherein at least one of the electrostimulation unit or the auxiliary electrostimulation unit comprises a power supply configured to provide power to both the electrostimulation unit and the auxiliary electrostimulation unit.

14. The apparatus of claim 1, comprising a temporary, at least partially elongated delivery member configured to deliver the electrostimulation electrode assembly to the target implant location, the delivery member sized, shaped, or otherwise configured to allow the delivery member to be detachably engaged with the electrostimulation electrode assembly.

15. A method, comprising:
providing an implantable medical device (IMD) configured to generate electrostimulation for stimulating first and second stimulation sites inside or on an surface of a heart of a subject via at least first and second electrodes;
providing the IMD to be positioned at a target implant location, including:
providing a temporary, at least partially elongated delivery member configured to attach to the IMD;
attaching the delivery member to the IMD and disposing the attached delivery member and the IMD to the target implant location;
providing a fixation element on the IMD, the fixation element configured to affix the at least first and second electrodes to the first and second stimulation sites using at least one fixation guide and at least one fixation member; and
releasing the delivery member from the IMD; and
delivering the electrostimulation to the first and second stimulation sites via the at least first and second electrodes.

16. The method of claim 15, wherein providing a fixation element includes using the at least one fixation guide to receive and retain a maneuvering device for positioning the IMD at the target implant location.

17. The method of claim 16, further comprising providing an extension cable configured to connect the at least first and second electrodes to the IMD, wherein providing a fixation element includes:
passing the maneuvering device through a lumen of the extension cable; and
using the maneuvering device to engage, steerably position, and secure the at least one electrode connected to the extension cable to one of the first and second stimulation sites.

18. The method of claim 15, wherein providing at least one fixation guide includes providing one or more bores, the one or more bores configured to connect to respective one or more hollow channels along an at least partially elongated housing of the IMD.

19. The method of claim 15, wherein delivering the electrostimulation to the first and second stimulation sites includes:
positioning the at least first and second electrodes on epicardial sites of at least two heart chambers; and
delivering the electrostimulations to the epicardial sites via the at least first and second electrodes.

20. The method of claim 15, wherein delivering the electrostimulation to the first and second stimulation sites includes:
positioning the at least first and second electrodes on endocardial sites of at least two heart chambers; and
delivering the electrostimulations to the endocardial sites via the first and second electrodes.
